(19) United States
(12) Patent Application Publication
(10) Pub. No.: US 2012/0172799 A1
(43) Pub. Date: Jul. 5, 2012

(54) DISPOSABLE SAFETY SYRINGE WITH RETRACTABLE NEEDLE TIP

(76) Inventor: Bo Jiang, Zhanjiang Province (CN)

(21) Appl. No.: 13/395,402

(22) PCT Filed: Apr. 16, 2010
(86) PCT No.: PCT/CN10/00520

§ 371 Date: Mar. 9, 2012

(30) Foreign Application Priority Data
Sep. 10, 2009 (CN) CN200920310057.6
Sep. 22, 2009 (CN) CN200920311017.3

(51) Int. Cl. A61M 5/50

(52) U.S. Cl. 604/110

(57) ABSTRACT

The invention discloses a safety syringe with a retractable needle tip, which includes a barrel (1), a needle seat (2) mounted in a mounting hole (11) at the head part of the barrel (1), and a core rod (3) with a snap-fit structure, wherein a stop shoulder structure is arranged at the upper part on the outer side of the needle seat (2) and the upper side wall of the mounting hole (11) of the barrel (1), and the barrel (1) and the needle seat (2) are positioned by means of the stop shoulder structure; a self-destruction blocking bar (5) for positioning and stopping the needle seat (2) is mounted at the bottom of the needle seat (2); and the head part of the core rod (3) is in snap-fit connection with the top end of the self-destruction blocking bar (5). The invention relates to a disposable safety syringe with retractable needle tip, of which, the needle seat and barrel position precisely, the syringe seat provides strong and stable injection support force, the needle seat pulls backward easily, and the locking structure of the barrel and the core rod locks effectively, and thus fundamentally overcomes difficulty in assembly of the needle seat and the barrel and prevents the core rod from being pulled out of or re-pushed into the cavity of the barrel.

### TECHNICAL FIELD

The invention relates to a disposable safety syringe, in particular to a disposable safety syringe with a retractable needle tip.

### BACKGROUND OF THE INVENTION

In order to avoid cross-infection when a medium is inputted into or taken out of a human body or an animal, a hypodermic syringe must be disposable and needs to be disposed after use. At present, the disposable syringe which is most widely used includes a barrel, a needle and a fixed-stroke piston assembled in the barrel. The biggest problem of syringes of this type is that, the needle remains exposed outside after injection, thereby not only bringing inconvenience to transportation and destruction treatment of the syringe waste, but also possibility of pricking medical staff due to inadvertence, and hence causing secondary cross-infection of pathogenic bacteria and becoming a hidden medical hazard not to be ignored. Therefore, the disposable safety syringe with retractable needle emerges. For example, Chinese patent CN1803212 discloses ‘a safety self-destruction medical syringe’, the retraction of whose needle tip is realized by manually pulling a needle seat backwards with a core rod, and the positioning of the needle seat and the barrel of the syringe is realized by an annular shoulder on the outer side of the needle seat and an annular clamping groove on the barrel; in order to avoid exposure of the needle caused by pulling the core rod out of the barrel with excess of force or by pushing back the core rod and causing the needle to pierce the barrel, a clamping ring on the core rod and an elastic convex jaw arranged at an annular position-clamping ring at the bottom of the barrel are in snap-fit connection correspondingly so as to deadly lock the head part of the core rod at the bottom of the barrel. The structural design is defective in several manners. On the one hand, the connection method results in difficulty of positioning of the needle seat in the barrel, and of assembly, and in possible assembly failure caused by slipping off of the annular shoulder out of the annular clamping groove due to excess of force; while during the injection process, the injection support force of the needle seat is not strong and stable enough, so that the needle seat might rotate and great discomfort is brought to a patient; in addition, manual backward pulling of the needle seat after completion of injection needs to overcome resistance of the snap-fit connection between the annular shoulder and the annular clamping groove, which is effort-consuming and difficult. Furthermore, as the elastic convex jaw is easy to deform, the locking effect is poor, and once the pulling or pushing force of the core rod exceeds a certain limit, the core rod can still be pulled out of or be re-pushed into the barrel so as to cause the exposure of the needle tip, namely the self-destruction failure of the syringe. Self-destruction failure of syringes is not only a potential safety hazard for waste disposal, but also a loophole for lawless persons to reuse them. Therefore, a disposable safety syringe which is accurate in positioning of the barrel and the needle seat with a tight locking between the core rod and the barrel, and which can truly realize thorough self-destruction after use, needs to be designed.

### SUMMARY OF THE INVENTION

The invention aims at providing a disposable safety syringe with a retractable needle tip, of which, the needle seat and barrel position precisely, the syringe seat provides strong and stable injection support force, the needle seat pulls backward easily, and the locking structure of the barrel and the core rod locks effectively, and can fundamentally overcomes difficulty in assembly of the needle seat and the barrel and prevents the core rod from being pulled out of or re-pushed into the cavity of the barrel.

The technical scheme for realizing the purpose of the invention is as follows: the safety syringe with the retractable needle tip includes a barrel, a needle seat mounted in a mounting hole at the head part of the barrel and a core rod with a snap-fit structure, wherein a stop shoulder structure is arranged at the upper part on the outer side of the needle seat and the upper side wall of the mounting hole of the barrel, and the barrel and the needle seat are positioned by means of the stop shoulder structure; a self-destruction blocking bar for positioning and stopping the needle seat is mounted at the bottom of the needle seat; and the head part of the core rod is in snap-fit connection with the top end of the self-destruction blocking bar.

A fixing recess for mounting the self-destruction blocking bar is arranged at the bottom of the needle seat.

A longitudinally through inner hole is arranged in the body of the self-destruction blocking bar, the head part of the body reaches the equal-diameter sliding section at the lower part of an inner hole of the needle seat, a convex ring is simultaneously arranged at the top end of the head part of the body of the self-destruction blocking bar, the convex ring can be in snap-fit connection with a bulge at the bottom of the equal-diameter sliding section of the needle seat, and the convex ring of the self-destruction blocking bar remains a certain distance from the bulge of the needle seat before self-destruction occurs; and at least two support legs are arranged at the bottom of the body of the self-destruction blocking bar, each support leg extends out of the needle seat and is respectively in snap-fit connection with clamping grooves arranged on the lower side wall of the mounting hole of the barrel.

The inner hole of the self-destruction blocking bar is partitioned into a small inner-diameter slope hole section, an equal inner-diameter connecting hole section and a large inner-diameter slope hole section from top to bottom. All the support legs of the self-destruction blocking bar radiate uniformly out of the center of the body.

A bending groove is arranged at the joint of each support leg of the self-destruction blocking bar and the body of the self-destruction blocking bar respectively.

The stop shoulder structure includes an upper shoulder arranged on the mounting hole of the barrel and a lower shoulder arranged on the outer side of the needle seat.

The upper shoulder of the stop shoulder structure is used for partitioning the mounting hole of the barrel into a upper small-diameter hole section and a lower large-diameter hole section and the clamping grooves are arranged on the lower large-diameter hole section.

The lower shoulder of the stop shoulder structure is used for partitioning the outer side of the needle seat to a small-diameter upper section and a large-diameter lower section, wherein the small-diameter upper section fits with the upper small-diameter hole section of the barrel, and the large-diameter lower section fits with the lower large-diameter hole section of the barrel.

An annular groove is arranged on the large-diameter lower section of the lower shoulder and an O-shaped ring is arranged in the annular groove.

A snap-fit convex ring is arranged at the tail part of the cavity of the barrel, and the upper side surface and the lower side surface of the snap-fit convex ring are arranged being vertical to the wall of the cavity of the barrel; and an annular upper flange and a lower flange are arranged on the body of the core rod with a space therebetween, wherein the outer diameter of each of the upper flange and the lower flange is greater than the inner diameter of the snap-fit convex ring of the barrel and fits the diameter of the cavity of the barrel, the lower flange can deform and climb over the snap-fit convex ring when the core rod is pulled backwards till the tail part of the cavity of the barrel, while the upper flange is stuck with the upper side surface of the snap-fit convex ring.

The body of the core rod includes an upper body and a lower body, which are in snap-fit connection, the upper flange is arranged on the upper body and the lower flange is arranged on the lower body.

The external surface of the lower flange of the core rod is in a slope structure which runs obliquely inward from top to bottom.

A snap-fit circumferential edge arranged on the outer side of the upper end surface of the lower flange of the core rod is higher than the upper end surface.

The external surface of the upper flange of the core rod is in a slope structure which runs obliquely outward from top to bottom.

A rounded structure is arranged between the upper side surface of the snap-fit convex ring of the barrel and the inner side surface thereof.

The safety syringe with the retractable needle tip further includes a funnel type mounting ring; and the mounting ring includes a body part connected with the barrel and a base plate, wherein the outer diameter of the body part fits the cavity of the barrel at the lower part of the snap-fit convex ring of the barrel, the upper section of the inner hole of the body part is an isodiametric section, the diameter of the upper section of the inner hole of the body part is equivalent to the inner diameter of the snap-fit convex ring of the barrel and the lower section of the inner hole of the body part is a large straight surface chamfer with increasing diameters.

A pushing plate for injection is arranged at the tail part of the body of the core rod by tight fit, wherein the outer diameter of a connecting part at the tail part of the body, which is used for connecting with the pushing plate, is smaller than the isodiametric inner port upper section of the mounting ring.

After adopting the above technical scheme, the invention has the following beneficial effects: (1) during assembling, the self-destruction blocking bar is firstly mounted at the bottom of the needle seat, then the integrated self-destruction blocking bar and the needle seat are placed in from the rear part of the barrel till the lower shoulder on the outer side of the needle seat is engaged with the upper shoulder in the mounting hole of the barrel and thus stopped, then the needle seat is rotated so as to enable all the support legs of the self-destruction blocking bar to be aligned with the clamping grooves on the barrel, and all the support legs enter into corresponding clamping grooves through forced pushing for realizing mounting positioning of the needle seat. The stop shoulder structure has dual functions of stopping and acting as a reference during the mounting process herein, thereby effectively avoiding the occurrence of mounting dislocation phenomenon caused by excess of force during the mounting of the needle seat, simplifying the assembly process, and improving assembling efficiency. The head part of the core rod with the snap-fit structure runs through the inner hole of the self-destruction blocking bar from bottom to top so as to realize snap-fit connection with the top end of the head part of the body of the self-destruction blocking bar, thus achieving good locking effect.

(2) After the mounting of the needle seat is completed, the positioning is realized through cooperation of the stop shoulder structure and the self-destruction blocking bar. The connection between the whole barrel and the needle seat is compact, with no clearance, and the positioning is very stable. During the injection process, the needle seat acts with even force on all the support legs of the self-destruction blocking bar, but not on the body of the self-destruction blocking bar directly, therefore, with no displacement or deformation during the process, the self-destruction blocking bar provides strong and stable support force to the needle seat; simultaneously, the needle seat is rotated, the plurality of the support legs of the self-destruction blocking bar and the clamping grooves of the barrel are in snap-fit connection respectively, and the structure fundamentally avoids rotation of the needle seat during the injection process and mitigates suffering of a patient.

(3) Special fixing recesses are arranged on the needle seat of the invention, for better mounting effect of the self-destruction blocking bar. The syringe has five specifications, namely 0.5 ml, 1 ml, 3 ml, 5 ml and 10 ml during actual production. As the needle seats of 0.5 ml syringe and 1 ml syringe are relatively small and the needle is generally fixed on the needle seat and does not need to be replaced, the support legs of the self-destruction blocking bar do not need to be in snap-fit connection with the clamping grooves of the barrel for preventing the needle seat from rotating during actual usage, and the self-destruction blocking bar can be directly inserted into the needle seat for simpler processing of the needle seat and more convenient assembly of the needle seat and the barrel; and uniform fixing recess can be arranged of course. Meanwhile, the structure is not only limited to the needle seat of 0.5 ml syringe or 1 ml syringe, and the needle seat structure can also be adopted by the large-specification barrel if the structure of the head part is small.

(4) In order to ensure that the head part of the core rod can smoothly go through the inner hole of the self-destruction blocking bar, the inner hole of the self-destruction blocking bar is partitioned into the small inner-diameter slope hole section, the equal inner-diameter connecting hole section and the large inner-diameter slope hole section from top to bottom. When the core rod is pulled backwards, the body of the self-destruction blocking bar is directly stressed, but no support leg is directly stressed, hence the body of the self-destruction blocking bar will move down synchronously along with the core rod and all the support legs can be folded up relative to the body and be separated from the respective clamping grooves on the barrel so as to release positioning constraint on the bottom of the needle seat. As the flange of the self-destruction blocking bar is separated from the bulge of the needle seat by the certain distance before self-destruction occurs, at the beginning of synchronous downward movement of the body of the self-destruction blocking bar under the drive of the core rod, only the body of the self-destruction blocking bar slides down relative to the isodiametric sliding section of the cavity of the needle seat, and all the support legs are folded up relative to the body and separated from the respective clamping grooves of the barrel, with the needle seat unmoved. The process continues till the flange of the self-destruction blocking bar is in snap-fit connection with the bulge at the bottom of the isodiametric sliding section of the needle seat, at which time, the snap-fit connection between all the support legs of the self-destruction blocking bar and the clamping grooves of the barrel is completely released, namely the constraint on backward movement of the needle seat does not exist any more, and the needle seat can move backwards together with the self-destruction blocking bar and the core rod in a very easy and convenient manner so as to drive the needle to completely enter into the barrel for realizing self-destruction.

(5) In order to enhance the effect of folding up all the support legs when the self-destruction blocking bar moves backwards under the action of the core rod and ensure smooth release of the constraint on all the support legs and the barrel, a bending groove is arranged at the joint of each support leg of the self-destruction blocking bar with the body respectively and the structural design can effectively increase the deformation amount of each support leg relative to the body.

(6) The upper side surface and the lower side surface of the snap-fit convex ring of the barrel are both arranged being vertical to the cavity wall of the barrel, the outer diameter of either the upper flange or the lower flange of the core rod is larger than the inner diameter of the snap-fit convex ring of the barrel; the external surface of the upper flange of the core rod is in the slope structure which is obliquely outward from top to bottom, the external surface of the lower flange is in the slope structure which is obliquely inward from top to bottom; thus, when the core rod is pulled backwards, an inverted conical surface structure formed by the external surface of the lower flange can enable the lower flange to deform and climb over the snap-fit convex ring of the barrel, to be reset smoothly and stuck with the lower side surface of the snap-fit convex ring; simultaneously, a positive conical surface structure formed by the external surface of the upper flange can not deform and is stuck with the upper side surface of the snap-fit convex ring, namely the snap-fit convex ring is completely stuck by the upper flange and the lower flange, and the core rod can neither be pulled backwards nor pushed upwards without damaging the structures of the upper flange, the lower flange or the snap-fit convex ring; thus the structural design effectively overcomes the defect of incomplete snap-fit self-destruction of the core rod and the barrel of prior art, and fundamentally avoids exposure of the needle tip caused by improper treatment, ensures self-destruction effect, brings convenience to waste disposal treatment, and simultaneously prevents the syringe from being re-used by lawless persons.

(7) In order to ensure that the lower flange can smoothly climb over the snap-fit convex ring of the barrel when the core rod is pulled backwards, a small rounded structure is arranged between the upper side surface and the inner side surface of the snap-fit convex ring of the barrel. At the same time, in order to enhance deformation capability of the lower flange, a snap-fit circumferential edge which is higher than the upper end surface is arranged on the outer side of the upper end surface of the lower flange of the core rod, so as to effectively enhance the contraction ability of the lower flange. However, the protruding snap-fit circumferential edge structure brings difficulty to demoulding of the upper flange and the lower flange from each other during manufacture; in view of this, the body of the core rod in the invention is arranged into two parts, namely the upper body and the lower body, which are in snap-fit connection, the upper flange being arranged on the upper body and the lower flange being arranged on the lower body, thus solving the demoulding problem during processing.

(8) The locking mechanism of the core rod and the barrel can realize self-destruction locking of the core rod and the barrel, but at the same time brings difficulty to assembly of the core rod and the barrel; if the two are directly mounted, the upper flange of the core rod can deform and climb over the snap-fit convex ring of the barrel while inserting the core rod into the barrel, but the lower flange of the core rod can not deform and is stuck with the lower side surface of the snap-fit convex ring of the barrel; in view of the above, the invention designs the funnel type mounting ring; during assembling, the body part of the mounting ring is firstly inserted into the tail part of the barrel, and then the core rod is extruded into the large chamfer with straight surface from the lower section of the inner hole of the body part of the mounting ring so as to enable the upper flange and the lower flange of the core rod to deform simultaneously, to enter into the isodiametric upper section of the cavity of the body part of the mounting ring, further to enter into the cavity of the snap-fit convex ring of the barrel and finally to smoothly enter into the barrel for realizing mounting; at this time, the pushing plate arranged at the tail part of the body of the core rod is detached, then the mounting ring can be taken out for repeated utilization, and the pushing plate is further placed back to complete the whole assembly process.

### BRIEF DESCRIPTION OF THE DRAWINGS

In order to make the invention to be understood more easily and clearly, the invention is further described in detail with combination of embodiments and figures.

FIG. 1 is a schematic planar diagram of the invention;

FIG. 2 is an enlargement of position A in FIG. 1 of the invention;

FIG. 3 is a sectional planar view of the barrel of the invention;

FIG. 4 is a sectional planar view of the needle seat of the invention;

FIG. 5 is a sectional planar view of another structure of the needle seat of the invention;

FIG. 6 is a sectional planar view of the self-destruction blocking bar of the invention;

FIG. 7 is a top view of FIG. 6 of the invention;

FIG. 8 is a schematic layout diagram of the self-destruction blocking bar with three support legs of the invention;

FIG. 9 is a schematic layout diagram of the self-destruction blocking bar with four support legs of the invention;

FIG. 10 is a schematic planar diagram of the self-destruction blocking bar of the invention with its support legs folded up;

FIG. 11 is a schematic diagram of the core rod of the invention;

FIG. 12 is a schematic diagram of another structure of the core rod of the invention;

FIG. 13 is a schematic diagram of fit between the barrel and core rod of the invention before self-destruction;

FIG. 14 is a schematic diagram of self-destruction snap-fit fit between the barrel and core rod of the invention;

FIG. 15 is an enlargement in position B in FIG. 14 of the invention;

FIG. 16 is a schematic diagram in split type of the core rod of the invention;

FIG. 17 is a schematic diagram of fit between the funnel type mounting ring and barrel of the invention.

Reference numbers in the figures are as follows:
barrel 1, mounting hole 11, upper shoulder 111, upper small-diameter hole section 112, lower large-diameter hole section 113, clamping groove 1131, snap-fit convex ring 12, upper surface 121, lower surface 122, inner side surface 123, rounded structure 124, cavity 13 and cavity wall 131;
needle seat 2, lower shoulder 21, small-diameter upper section 22, large-diameter lower section 23, annular groove 231, isodiametric sliding section 24, bulge 25 and fixing recess 26;
core rod 3, upper flange 31, upper flange external surface 311, lower flange 32, lower flange external surface 321, snap-fit circumferential edge 322, pushing plate 33 and connecting part 331;
O-shaped ring 4;
self-destruction blocking bar 5, body 51, inner hole 511, small inner-diameter slope hole section 5111, equal inner-diameter connecting hole section 5112, large inner-diameter slope hole section 5113, projecting ring 512, support legs 52 and bending groove 53;
mounting ring 6, body part 61, base plate 62, inner port upper section 63 and inner port lower section 64.

### DETAILED DESCRIPTION OF THE INVENTION

### Embodiment 1

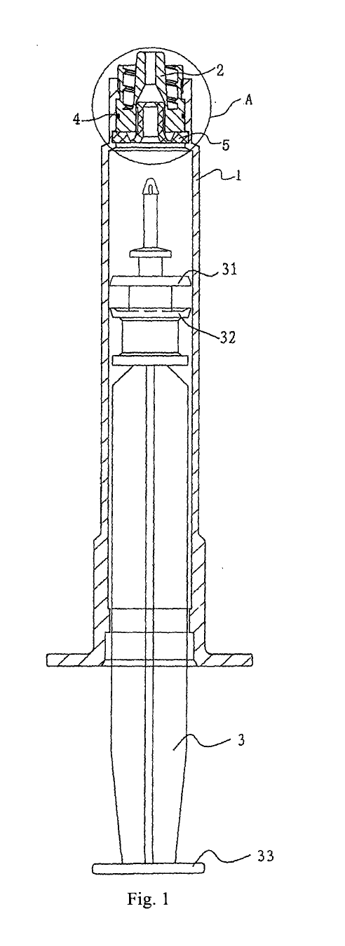

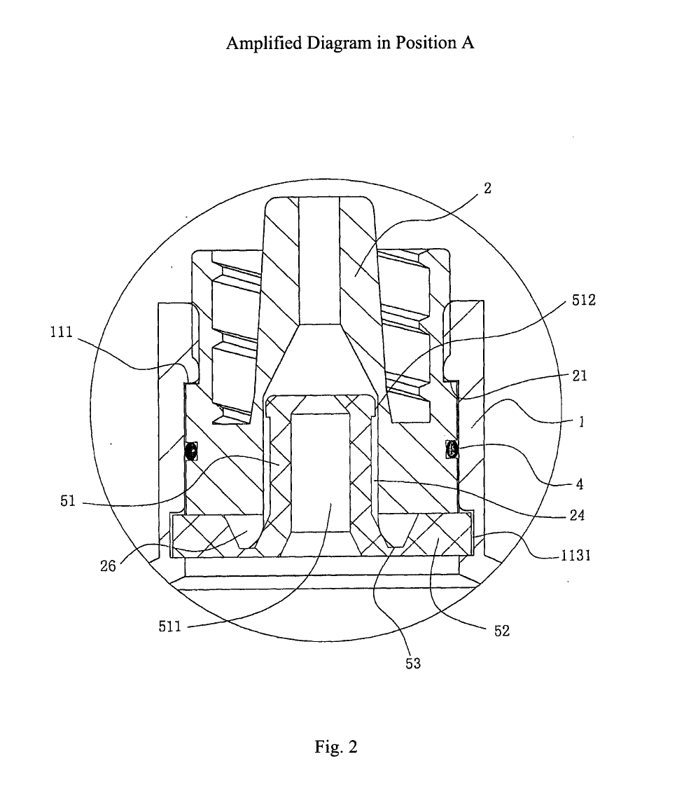

As shown in FIG. 1 and FIG. 2, a safety syringe with a retractable needle tip of the embodiment includes a barrel 1, a needle seat 2 mounted in a mounting hole 11 at the head part of the barrel 1 and a core rod 3 with a snap-fit structure. A stop shoulder structure is arranged at the external upper part of the needle seat 2 and the upper side wall of the mounting hole 11 of the barrel 1, and the barrel 1 and the needle seat 2 are positioned by means of the stop shoulder structure; a fixing recess 26 is arranged at the bottom of the needle seat 2 and a self-destruction blocking bar 5 for positioning and stopping the needle seat 2 is further mounted at the bottom of the needle seat 2; and the head part of the core rod 3 is in snap-fit connection with the top end of the self-destruction blocking bar 5.

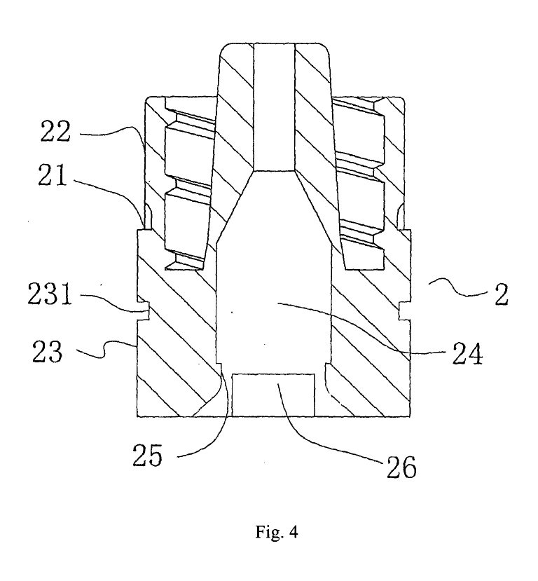

As shown in FIG. 1 to FIG. 4, the stop shoulder structure includes an upper shoulder 111 arranged on the mounting hole 11 of the barrel 1 and a lower shoulder 21 arranged on the outer side of the needle seat 2. The upper shoulder 111 of the stop shoulder structure is used for partitioning the mounting hole 11 of the barrel 1 into an upper small-diameter hole section 112 and a lower large-diameter hole section 113, with the clamping groove 1131 arranged on the lower large-diameter hole section 113. The lower shoulder 21 of the stop shoulder structure is used for partitioning the outer side of the needle seat 2 to a small-diameter upper section 22 and a large-diameter lower section 23, wherein the small-diameter upper section 22 fits with the upper small-diameter hole section 112 of the barrel 1, and the large-diameter lower section 23 fits with the lower large-diameter hole section 113 of the barrel 1. An annular groove 231 is arranged on the large-diameter lower section 23 of the lower shoulder 21 and an O-shaped ring 4 is arranged in the annular groove 231.

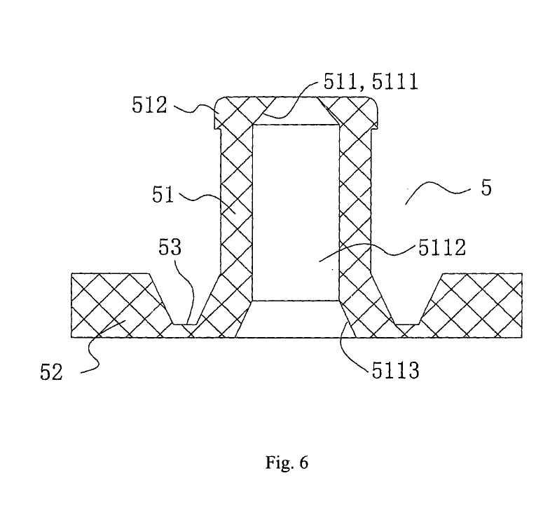

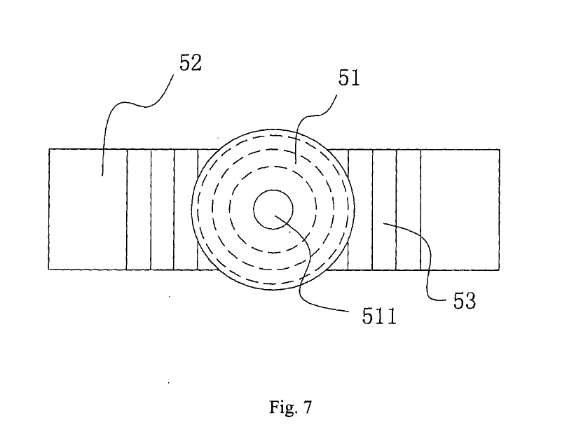

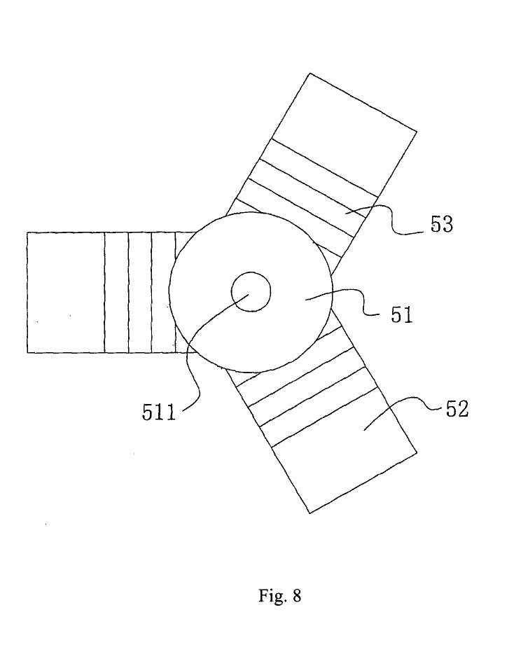

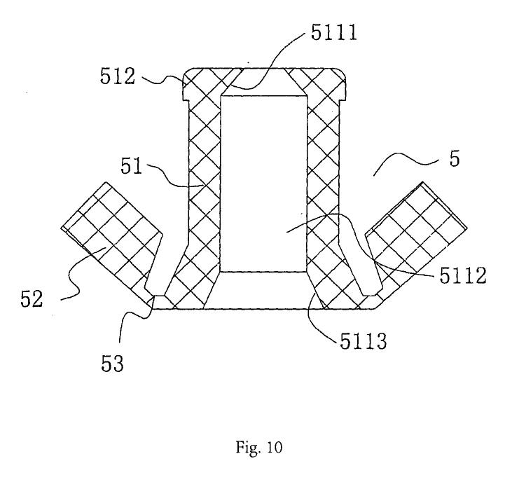

As shown in FIG. 6 to FIG. 8, a longitudinally through inner hole 511 is arranged in the body 51 of the self-destruction blocking bar 5, the head part of the body 51 goes deep into an equal-diameter sliding section 24 at the lower part of the inner hole of the needle seat 2, a projecting ring 512 is simultaneously arranged at the top end of the head part of the body 51 of the self-destruction blocking bar 5, the projecting ring 512 can be in snap-fit connection with a bulge 25 at the bottom of the equal-diameter sliding section 24 of the needle seat 2, and the projecting ring 512 of the self-destruction blocking bar 5 is separated from the bulge 25 of the needle seat 2 by a certain distance before self-destruction occurs. Three support legs 52 are arranged at the bottom of the body 51 of the self-destruction blocking bar 5, each support leg 52 extends out of the needle seat 2 and are respectively in snap-fit connection with clamping grooves 1131 arranged on the lower side wall of the mounting hole 11 of the barrel 1; and the support legs 52 of the self-destruction blocking bar 5 radiate uniformly out of the center of the body 51. A bending groove 53 is arranged at the joint of each support leg 52 of the self-destruction blocking bar 5 with the body 51 of the self-destruction blocking bar 5 respectively, and the state where the support legs 52 are folded up from the bending grooves 53 is as shown in FIG. 10. The inner hole 511 of the self-destruction blocking bar 5 is partitioned into a small inner-diameter slope hole section 5111, an equal inner-diameter connecting hole section 5112 and a large inner-diameter slope hole section 5113 from top to bottom so as to ensure that the head part of the core rod 3 can smoothly go into the inner hole 511 of the self-destruction blocking bar 5.

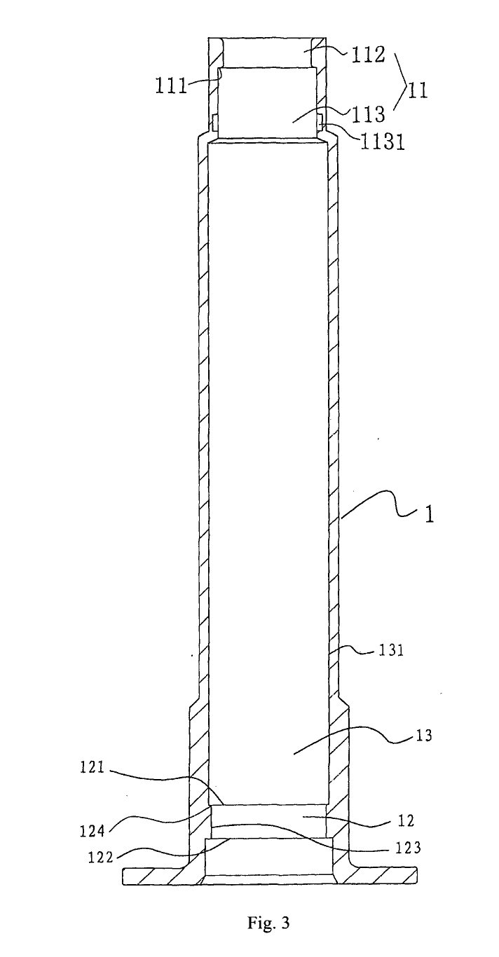

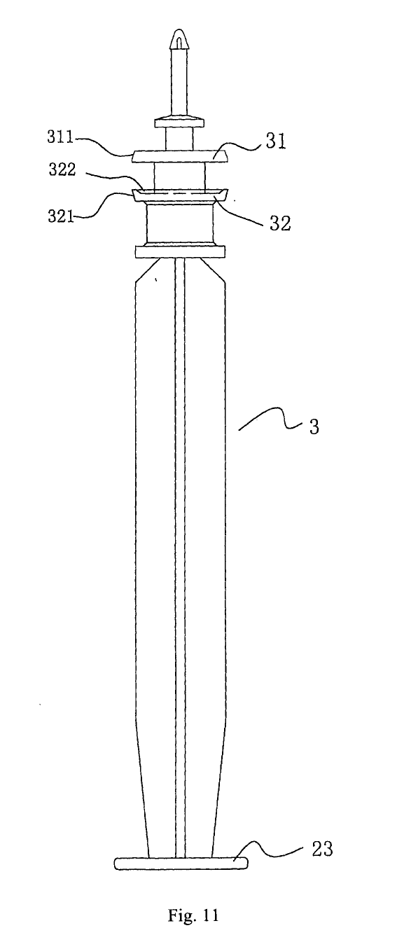

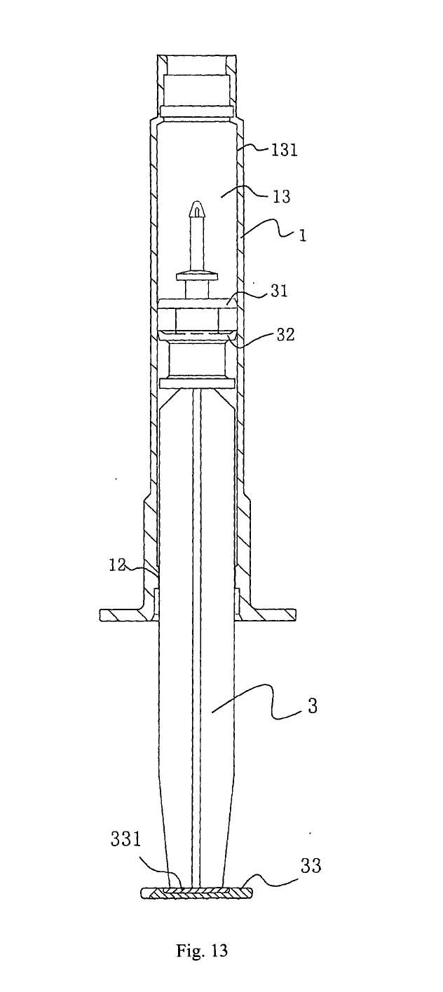

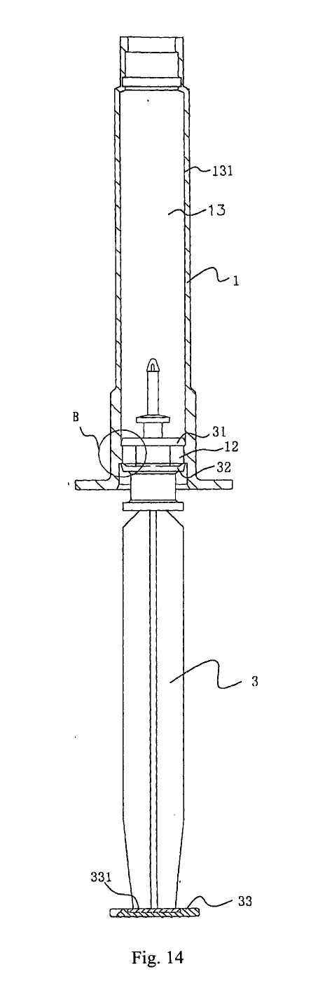

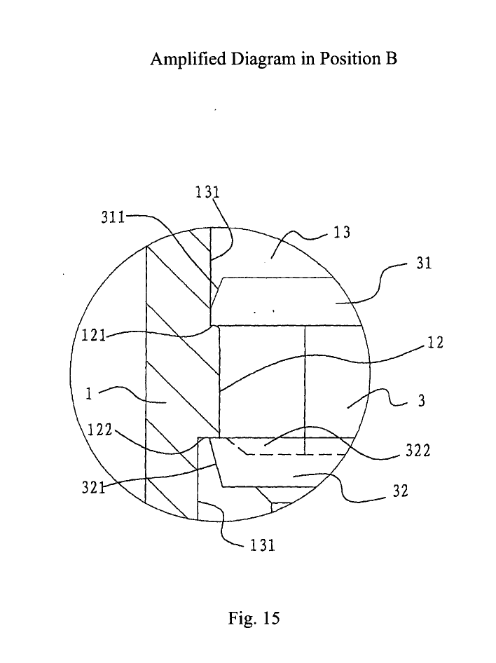

As shown in FIG. 3, a snap-fit convex ring 12 is arranged at the tail part of the cavity 13 of the barrel 1, and the upper surface 121 and the lower surface 122 of the snap-fit convex ring 12 are both arranged being vertical to the cavity wall 131 of the barrel 1. A rounded structure 124 is arranged between the upper surface 121 of the snap-fit convex ring 12 of the barrel 1 and the inner side surface 123 thereof. As shown in FIG. 11, an annular upper flange 31 and a lower flange 32 are arranged on the body of the core rod 3 with a space therebetween, wherein the outer diameter of either the upper flange 31 or of the lower flange 32 is greater than the inner diameter of the snap-fit convex ring 12 of the barrel 1, and can fit the diameter of the cavity 13 of the barrel 1, the lower flange 32 can deform and climb over the snap-fit convex ring 12 when the core rod 3 is pulled backwards till the tail part of the cavity 13 of the barrel 1, while the upper flange 31 is mutually stuck with the upper side surface of the snap-fit convex ring 12. In order to ensure that the upper flange 31 of the core rod 3 can deform and smoothly climb over the snap-fit convex ring 12 of the barrel 1 during mounting of the core rod 3 and the barrel 1, the external surface 321 of the lower flange 32 of the core rod 3 is in a slope structure which is obliquely inward from top to bottom; simultaneously, in order to ensure that the lower flange 32 of the core rod 3 can deform and climb over the snap-fit convex ring 12 of the barrel 1 during self-destruction, the external surface 311 of the upper flange 31 of the core rod 3 is in the slope structure which is obliquely outward from top to bottom. A snap-fit circumferential edge 322 which is higher than the upper end surface is arranged on the outer side of the upper end surface of the lower flange 32 of the core rod 3. The schematic diagrams of the barrel 1 and the core rod 3 before self-destruction and the snap-fit structure are as shown in FIG. 13-FIG. 15.

### Embodiment 2

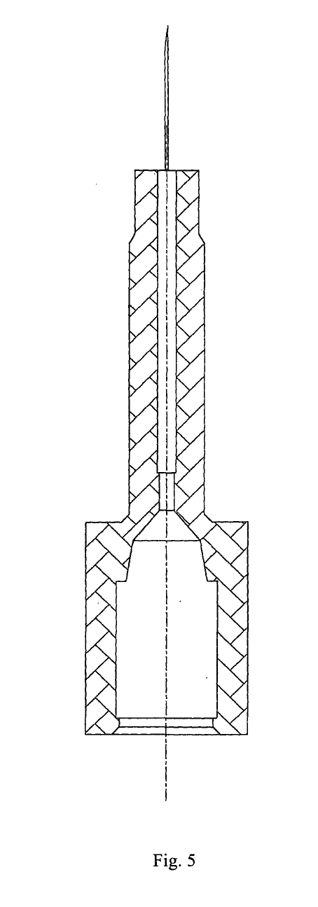

As shown in FIG. 5, the structure of the embodiment is basically the same with that of the embodiment 1, with difference that the fixing recess 26 is not arranged at the bottom of the needle seat 2; the structure is currently applicable to syringes of 0.5 ml and 1 ml, as well as to those with needle seat structures geared for large-specification needles with small head part structures.

### Embodiment 3

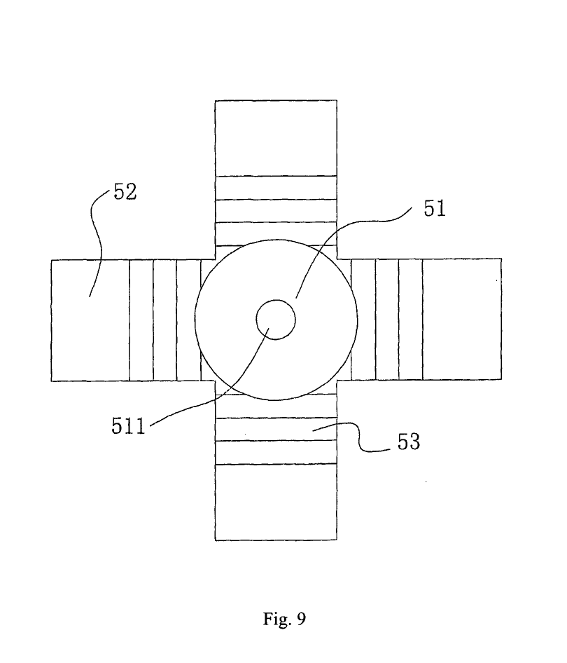

As shown in FIG. 9, the structure of the embodiment is basically the same with that of the embodiment 1, with the difference that the self-destruction blocking bar 5 has four support legs 52. The number of the support legs 52 is not limited and arbitrary plural support legs 52 can be arranged to radiate uniformly out of the center of the body 51.

### Embodiment 4

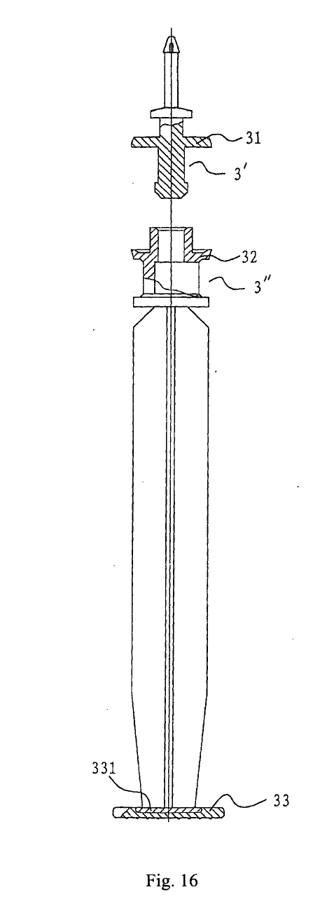

As shown in FIG. 16, the structure of the embodiment is basically the same with that of the embodiment 1, with the following differences: the body of the core rod 3 includes an upper body 3′ and a lower body 3″, which are in snap-fit connection, the upper flange 31 is arranged on the upper body 3′ and the lower flange 32 is arranged on the lower body 3″. As the protruding snap-fit circumferential edge structure brings difficulty to demoulding of the upper flange from the lower flange during manufacture, the structure of arranging the body of the core rod into two parts is adopted, namely the body of the core rod includes the upper body and the lower body, which are in snap-fit connection, the upper flange is arranged on the upper body and the lower flange is arranged on the lower body, so as to well solve the demoulding problem during manufacture.

### Embodiment 5

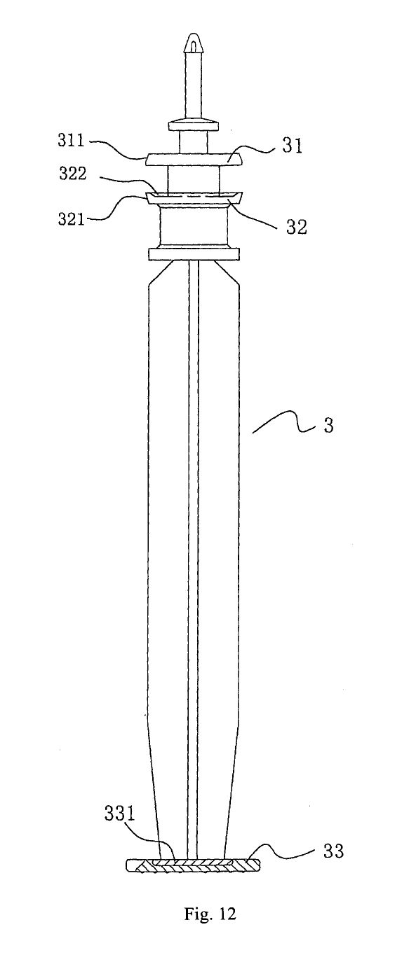

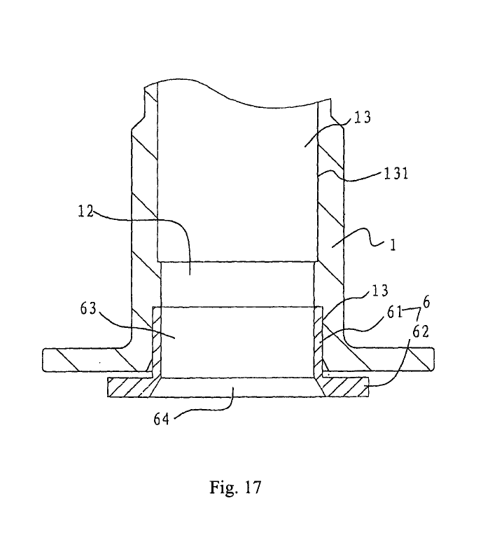

As shown in FIG. 12 and FIG. 17, the structure of the embodiment is basically the same with that of the embodiment 1, with the difference that the embodiment further includes a funnel type mounting ring 6; and the mounting ring 6 includes a body part 61 connected with the barrel 1 and a base plate 62, wherein the outer diameter of the body part 61 fits the cavity 13 of the barrel 1 at the lower part of the snap-fit convex ring 12 of the barrel 1, the inner port upper section 63 of the body part 61 is an isodiametric section, the diameter of which is equivalent to the inner diameter of the snap-fit convex ring 12 of the barrel 1, and the inner port lower section 64 of the body part 61 is a straight surface large chamfer with increasing diameters. In order to facilitate the use of the mounting ring 6, a pushing plate 33 for injection is arranged at the tail part of the body of the core rod 3 by tight fit, wherein the outer diameter of a connecting part 331 at the tail part of the body, which is used for connecting with the pushing plate 33, is smaller than the inner port isodiametric upper section 63 of the mounting ring 6.

The above-mentioned embodiments further describe the purpose, the technical scheme and the beneficial effects of the invention in detail. It should be understood that they are only the specific embodiments of the invention and are not used for limiting the invention, and all the modification, equivalent replacements, improvements and the like within the spirit and principle of the invention should be considered within the protection range of the invention.

1. A safety syringe with a retractable needle tip, comprising a barrel (1), a needle seat (2) mounted in a mounting hole (11) at the head part of the barrel (1) and a core rod (3) with a snap-fit structure, characterized in that a stop shoulder structure is arranged at the upper part on the outer side of the needle seat (2) and at the upper side wall of the mounting hole (11) of the barrel (1), and the barrel (1) and the needle seat (2) are positioned through the stop shoulder structure; a self-destruction blocking bar (5) for positioning and stopping the needle seat (2) is mounted at the bottom of the needle seat (2); and the head part of the core rod (3) is in snap-fit connection with the top end of the self-destruction blocking bar (5).

2. The safety syringe with the retractable needle tip according to claim 1, characterized in that a fixing recess (26) for mounting the self-destruction blocking bar (5) is arranged at the bottom of the needle seat (2).

3. The safety syringe with the retractable needle tip according to claim 1, characterized in that a longitudinally through inner hole (511) is arranged in the body (51) of the self-destruction blocking bar (5), the head part of the body (51) goes deep into an isodiametric sliding section (24) at the lower part of the inner hole of the needle seat (2), a projecting ring (512) is simultaneously arranged at the top end of the head part of the body (51) of the self-destruction blocking bar (5), the projecting ring (512) can be in snap-fit connection with a bulge (25) at the bottom of the isodiametric sliding section (24) of the needle seat (2), and the projecting ring (512) of the self-destruction blocking bar (5) is separated from the bulge (25) of the needle seat (2) by a certain distance before self-destruction occurs; and at least two support legs (52) are arranged at the bottom of the body (51) of the self-destruction blocking bar (5), each support leg (52) extends out of the needle seat (2) and is respectively in snap-fit connection with clamping grooves (1131) arranged on the lower side wall of the mounting hole (11) of the barrel (1).

4. The safety syringe with the retractable needle tip according to claim 3, characterized in that the inner hole (511) of the self-destruction blocking bar (5) is partitioned into a small inner-diameter slope hole section (5111), an equal inner-diameter connecting hole section (5112) and a large inner-diameter slope hole section (5113) from top to bottom.

5. The safety syringe with the retractable needle tip according to claim 4, characterized in that all the support legs (52) of the self-destruction blocking bar (5) radiate uniformly out of the center of the body (51).

6. The safety syringe with the retractable needle tip according to claim 5, characterized in that a bending groove (53) is arranged at the joint of each support leg (52) of the self-destruction blocking bar (5) with the body (51) of the self-destruction blocking bar (5) respectively.

7. The safety syringe with the retractable needle tip according to claim 1, characterized in that the stop shoulder structure comprises an upper shoulder (111) arranged on the mounting hole (11) of the barrel (1) and a lower shoulder (21) arranged on the outer side of the needle seat (2).

8. The safety syringe with the retractable needle tip according to claim 7, characterized in that the upper shoulder (111) of the stop shoulder structure is used for partitioning the mounting hole (11) of the barrel (1) into an upper small-diameter hole section (112) and a lower large-diameter hole section (113), with the clamping grooves (1131) arranged on the lower large-diameter hole section (113).

9. The safety syringe with the retractable needle tip according to claim 8, characterized in that the lower shoulder (21) of the stop shoulder structure is used for partitioning the outer side of the needle seat (2) to a small-diameter upper section (22) and a large-diameter lower section (23), wherein the small-diameter upper section (22) fits with the upper small-diameter hole section (112) of the barrel (1), and the large-diameter lower section (23) fits with the lower large-diameter hole section (113) of the barrel (1).

10. The safety syringe with the retractable needle tip according to claim 9, characterized in that an annular groove (231) is arranged on the large-diameter lower section (23) of the lower shoulder (21) and an O-shaped ring (4) is arranged in the annular groove (231).

11. The safety syringe with the retractable needle tip according to claim 1, characterized in that a snap-fit convex ring (12) is arranged at the tail part of the cavity (13) of the barrel (1), and the upper side surface (121) and the lower side surface (122) of the snap-fit convex ring (12) are both arranged being vertical to the wall (131) of the cavity of the barrel (1); and an annular upper flange (31) and a lower flange (32) are arranged on the body of the core rod (3) with a space therebetween, wherein the outer diameter of either the upper flange (31) or the lower flange (32) is greater than the inner diameter of the snap-fit convex ring (12) of the barrel (1) and fits the diameter of the cavity (13) of the barrel (1), the lower flange (32) can deform and climb over the snap-fit convex ring (12) when the core rod (3) is pulled backwards till the tail part of the cavity (13) of the barrel (1), and the upper flange (31) is mutually stuck with the upper side surface of the snap-fit convex ring (12).

12. The safety syringe with the retractable needle tip according to claim 11, characterized in that the body of the core rod (3) comprises an upper body (3′) and a lower body (3″), which are in snap-fit connection, the upper flange (31) is arranged on the upper body (3′) and the lower flange (32) is arranged on the lower body (3″).

13. The safety syringe with the retractable needle tip according to claim 12, characterized in that the lower flange external surface (321) of the lower flange (32) of the core rod (3) is in a slope structure which is obliquely inward from top to bottom.

14. The safety syringe with the retractable needle tip according to claim 13, characterized in that a snap-fit circumferential edge (322) arranged on the outer side of the upper end surface of the lower flange (32) of the core rod (3) is higher than the upper end surface.

15. The safety syringe with the retractable needle tip according to claim 12, characterized in that the external surface (311) of the upper flange (31) of the core rod (3) is in the slope structure which is obliquely outward from top to bottom.

16. The safety syringe with the retractable needle tip according to claim 15, characterized in that a rounded structure (124) is arranged between the upper side surface (121) of the snap-fit convex ring (12) of the barrel (1) and the inner side surface (123) thereof.

17. The safety syringe with the retractable needle tip according to claim 12, characterized in that the safety syringe with the retractable needle tip further comprises a funnel type mounting ring (6); and the mounting ring (6) comprises a body part (61) connected with the barrel (1) and a base plate (62), wherein the outer diameter of the body part (61) fits the cavity (13) of the barrel (1) at the lower part of the snap-fit convex ring (12) of the barrel (1), the inner port upper section (63) of the inner hole of the body part (61) is an isodiametric section, the diameter of the inner port upper section (63) of the inner hole of the body part (61) is equivalent to the inner diameter of the snap-fit convex ring (12) of the barrel (1), and the inner port lower section (64) of the inner hole of the body part (61) is a straight surface large chamfer with increasing diameters.

18. The safety syringe with the retractable needle tip according to claim 17, characterized in that a pushing plate (33) for injection is arranged at the tail part of the body of the core rod (3) by tight fit, wherein the outer diameter of a connecting part (331) at the tail part of the body, which is used for connecting with the pushing plate (33), is smaller than the isodiametric inner port upper section (63) of the mounting ring (6).

19. The safety syringe with the retractable needle tip according to claim 2, characterized in that a longitudinally through inner hole (511) is arranged in the body (51) of the self-destruction blocking bar (5), the head part of the body (51) goes deep into an isodiametric sliding section (24) at the lower part of the inner hole of the needle seat (2), a projecting ring (512) is simultaneously arranged at the top end of the head part of the body (51) of the self-destruction blocking bar (5), the projecting ring (512) can be in snap-fit connection with a bulge (25) at the bottom of the isodiametric sliding section (24) of the needle seat (2), and the projecting ring (512) of the self-destruction blocking bar (5) is separated from the bulge (25) of the needle seat (2) by a certain distance before self-destruction occurs; and at least two support legs (52) are arranged at the bottom of the body (51) of the self-destruction blocking bar (5), each support leg (52) extends out of the needle seat (2) and is respectively in snap-fit connection with clamping grooves (1131) arranged on the lower side wall of the mounting hole (11) of the barrel (1).

20. The safety syringe with the retractable needle tip according to claim 2, characterized in that the stop shoulder structure comprises an upper shoulder (111) arranged on the mounting hole (11) of the barrel (1) and a lower shoulder (21) arranged on the outer side of the needle seat (2).
